SEQUENCE LISTING (8 sequences)

SEQ ID NO: 1 - protein, 21 aa, Artificial Sequence
  Synthetic polypeptide
AGYLLGKINLKACAALAKKCL

SEQ ID NO: 2 - protein, 21 aa, Artificial Sequence
  Synthetic polypeptide
AGYLLGKINLKASAALAKKSL

SEQ ID NO: 3 - protein, 21 aa, Artificial Sequence
  Synthetic polypeptide
ACYLLGKICLKALAALAKKIL

SEQ ID NO: 4 - protein, 21 aa, Artificial Sequence
  Synthetic polypeptide
AGYLLGKINLKALCALAKKIC

SEQ ID NO: 5 - protein, 21 aa, Artificial Sequence
  Synthetic polypeptide
  MISC_FEATURE (13)..(20): [Cys]C1=C(F)C(F)=C(C2=C(F)C(F)=C([Cys])C(F)=C2F)C(F)=C1F
  MISC_FEATURE (1)..(1): SEE SPECIFICATION
  AMIDATION (21)..(21): AMIDATION
AGYLLGKINLKACAALAKKCL

SEQ ID NO: 6 - protein, 21 aa, Artificial Sequence
  Synthetic polypeptide
  MISC_FEATURE (13)..(20): [Cys]C1=C(F)C(F)=C(C2=C(F)C(F)=C([Cys])C(F)=C2F)C(F)=C1F
  MISC_FEATURE (1)..(1): SEE SPECIFICATION
  AMIDATION (21)..(21): AMIDATION
AGYLLGKINLKACAALAKKCL

SEQ ID NO: 7 - protein, 21 aa, Artificial Sequence
  Synthetic polypeptide
  MISC_FEATURE (13)..(20): [Cys]C1=C(F)C(F)=C(C2=C(F)C(F)=C([Cys])C(F)=C2F)C(F)=C1F
  MISC_FEATURE (1)..(1): N[Pt](Cl)(OC(CCC(N[Ala])=O)=O)(OC(NCCCC)=O)(N)Cl
  AMIDATION (21)..(21): AMIDATION
AGYLLGKINLKACAALAKKCL

SEQ ID NO: 8 - protein, 21 aa, Artificial Sequence
  Synthetic polypeptide
  MISC_FEATURE (13)..(20): [Cys]C1=C(F)C(F)=C(C2=C(F)C(F)=C([Cys])C(F)=C2F)C(F)=C1F
  AMIDATION (21)..(21): AMIDATION
AGYLLGKINLKACAALAKKCL

(19) United States
(12) Patent Application Publication
(10) Pub. No.: US 2021/0023129 A1
(43) Pub. Date: Jan. 28, 2021

(54) PLATINUM PRODRUG PERFLUOROARYL PEPTIDE CONJUGATES

(71) Applicant: MASSACHUSETTS INSTITUTE OF TECHNOLOGY, Cambridge, MA (US)

(72) Inventors: Stephen Lippard, Washington, DC (US); Bradley Pentelute, Cambridge, MA (US); Omer Yilmaz, Cambridge, MA (US); Justin Wolfe, Cambridge, MA (US); Colin Fadzen, Boston, MA (US); Wen Zhou, Cambridge, MA (US)

(21) Appl. No.: 16/864,963

(22) Filed: May 1, 2020

Related U.S. Application Data
(60) Provisional application No. 62/842,815, filed on May 3, 2019.

(51) Int. Cl. A61K 33/243; A61K 47/64; A61P 35/00

(52) U.S. Cl. CPC A61K 33/243; A61P 35/00; A61K 47/64

(57) ABSTRACT

The disclosure provides platinum(IV) prodrug perfluoroaryl peptide conjugates that can be used to treat cell proliferative disorders, such as those associated with malignant tumor cells. The conjugates can improve the delivery of platinum(IV) prodrugs to glioma cells.

### RELATED APPLICATIONS

This application claims the benefit of U.S. Provisional Patent Application No. 62/842,815, filed May 3, 2019, the disclosure of which is incorporated by reference herein in its entirety.

### GOVERNMENT LICENSE RIGHTS

This invention was made with government support under Grant Nos. CA034992, AG045144, CA211184, and GM110535, awarded by the National Institutes of Health, Grant No. 1122374, awarded by the National Science Foundation Graduate Research Fellowship, and award number F30HD093358, awarded by the Eunice Kennedy Shriver National Institute of Child Health and Human Development of the National Institutes of Health. The government has certain rights in the invention.

### BACKGROUND

Glioblastoma (GBM) is the most common primary malignant brain tumor. Even with the current standard of care, which involves maximal safe surgical resection followed by radiation concurrent with temozolomide, the median survival from time of diagnosis is fifteen months. Radical resection of the primary mass is not curative because of infiltrating tumor cells present throughout the whole brain. These infiltrating cells enable recurrence of the disease. Although many experimental drugs have been tested in clinical trials for GBM over the past few decades, none have changed disease progression in any meaningful way. One limitation in the development of new drugs is poor drug delivery across the blood-brain tumor barrier (BBTB).

Platinum-based chemotherapeutics such as cisplatin, carboplatin, and oxaliplatin are widely used for the treatment of malignancies. Cisplatin for example, is a platinum(II) with two ammonia ligands and two chloride ligands. The mechanism of action for cisplatin involves exchange of a chloride ligand for water, followed by preferential platination of the nucleophilic N7 atom of a purine nucleobase. Another ligand substitution can occur with a nearby guanine base forming a DNA cross-link. The cells are arrested at the G2/M transition of the cell cycle in an attempt to repair the DNA lesion. If DNA repair is unsuccessful, the cells initiate apoptosis. The platinum-based chemotherapeutics also react with off-target nucleophiles, which can lead to significant toxic side effects such as nephrotoxicity.

One solution to mitigate off-target toxicity and improve the therapeutic index of platinum(II) chemotherapeutics is administering them as Pt(IV) prodrugs. The Pt(II) center of cisplatin is oxidized to Pt(IV) with the addition of two axial ligands. Upon internalization into cells, the Pt(IV) prodrug is reduced and enables the in situ generation of the native Pt(II) species inside a cancer cell. Unfortunately, premature reduction in the bloodstream can often limit the clinical utility of Pt(IV) prodrugs.

Accordingly, it would be desirable to have a composition and a method for delivering Pt(IV) prodrugs across the blood brain tumor barrier.

### SUMMARY

The disclosure provides a conjugate comprising a Pt(IV) prodrug covalently associated with a perfluororaryl peptide. The perfluoroaryl peptide could improve the delivery of Pt(IV) prodrugs to the tumor cell. In one embodiment, the tumor cell is a glioma cell.

In one embodiment, the conjugate is represented by the formula as follows (SEQ ID NO: 7):

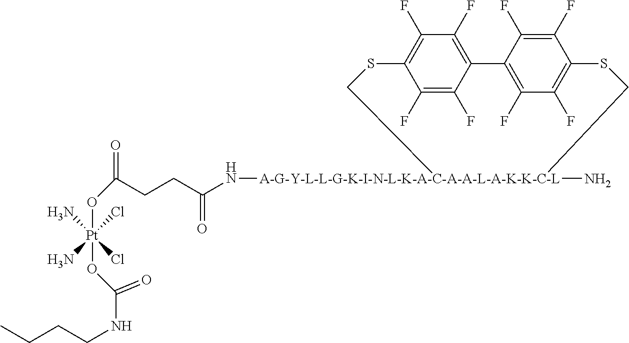

and pharmaceutically acceptable salts thereof.

The disclosure also provides a pharmaceutical composition comprising the Pt(IV) prodrug—perfluoroaryl peptide conjugates disclosed herein, or pharmaceutically acceptable salts thereof, in a pharmaceutically acceptable vehicle. In some embodiments, the compositions are formulated in a solution suitable for intravenous injection. In other embodiments, the compositions are formulated as oral, subcutaneous or intramuscular dosage forms.

The disclosure also provides methods for preparing the Pt(IV) prodrug—perfluoroaryl peptide conjugates disclosed herein.

The disclosure also provides methods of treating a disease or condition. The method comprises administering a therapeutically effective amount of the Pt(IV) prodrug perfluoroaryl peptide conjugates disclosed herein to a subject in need thereof. In one embodiment, the disease is glioblastoma.

Various embodiments, objects, features, and advantages will become more apparent from the following detailed description of the embodiments or may be learned by practice of the claimed invention. These objects and advantages will be realized and attained by the compositions and methods described and claimed herein. This summary section has been made with the understanding that it is to be considered as a brief and general synopsis of some of the embodiments disclosed herein, and is not intended to be used to limit the scope of the appended claims.

### BRIEF DESCRIPTION OF THE FIGURES

FIG. 1A shows the amino acid sequences of TP10 M13 (SEQ ID NO:1) and the linear control TP10 L13 (SEQ ID NO:2). FIG. 1B shows perfluoroaryl cyclization of Pt(IV)-prodrug functionalized peptide. FIG. 1C shows LC-MS analysis of the starting material, crude reaction, and purified material after reversed-phase high-performance liquid chromatography. All chromatograms are total ion currents (TIC).

FIG. 2A shows cell viability of G9 neurospheres treated with cisplatin, PtIV-M13, and prodrug 4 (PtIV). FIG. 2B shows cell viability of G30 neurospheres treated with cisplatin, PtIV-M13, and the linear control PtIV-L13. The error bars are the standard deviation of three replicates.

FIG. 3A shows the whole cell concentration of platinum of G9 GCs treated with 5 μM of cisplatin or the PtIV-M13 conjugate for 5 hours. The error bars represent the standard deviation of three replicate wells. FIG. 3B shows the concentration of platinum in cell components of G9 GSCs treated with 5 μM of cisplatin or the PtIV-M13 conjugate for 5 hours. The error bars represent the standard deviation of three different wells treated with the compound.

FIG. 4A shows the concentration of platinum in serum after treatment. FIG. 4B shows the concentration of platinum in the brain after treatment. FIG. 4C shows the concentration of platinum in the brain of mice 5 hours after treatment with either PtIV-M13, PtIV-L13, or cisplatin (n=4 animals for each treatment group). The results are significant with a p-value of less than 0.0001 by a One-Way ANOVA test.

### DETAILED DESCRIPTION

### Definitions

It is to be understood that the terminology used herein is for the purpose of describing particular embodiments only, and is not intended to be limiting. As used in this specification and the appended claims, the singular forms “a,” “an,” and “the” include plural referents unless the context clearly dictates otherwise. Thus, for example, reference to “an excipient” includes a combination of two or more such excipients, reference to “an active pharmaceutical ingredient” includes one or more active pharmaceutical ingredients, and the like. Unless specifically stated or obvious from context, as used herein, the term “or” is understood to be inclusive and covers both “or” and “and.”

Unless defined otherwise, all technical and scientific terms used herein have the same meaning as commonly understood by one of ordinary skill in the art to which the invention pertains. Although other methods, systems, and networks similar, or equivalent, to those described herein can be used in the practice of the present invention, the preferred materials and methods are described herein.

In describing and claiming the present invention, the following terminology will be used in accordance with the definitions set out below.

The terms “subject” and “patient” as used herein refers to any member of the subphylum Chordata, including, without limitation, humans and other primates, including non-human primates such as rhesus macaques and other monkey species and chimpanzees and other ape species; farm animals such as cattle, sheep, pigs, goats, and horses; domestic mammals such as dogs and cats; laboratory animals including rodents such as mice, rats, and guinea pigs; birds, including domestic, wild, and game birds such as chickens, turkeys, and other gallinaceous birds, ducks, geese, and the like. The term does not denote a particular age or gender. Thus, both adult and newborn individuals are intended to be covered.

The terms “administer” and “administering” as used herein refer to providing a therapeutic to a subject. Multiple techniques of administering a therapeutic exist in the art including, but not limited to, intravenous, oral, aerosol, parenteral, ophthalmic, pulmonary, and topical administration.

The terms “treatment” and “treating” as used herein refer to amelioration of a disease or disorder, or at least one sign or symptom thereof. “Treatment” or “treating” can refer to reducing the progression of a disease or disorder, as determined by, e.g., stabilization of at least one sign or symptom or a reduction in the rate of progression as determined by a reduction in the rate of progression of at least one sign or symptom. In another embodiment, “treatment” or “treating” refers to delaying the onset of a disease or disorder.

The term “therapeutically effective amount” as used herein refers to an amount of a drug, formulation, or composition to achieve a particular biological result. In certain embodiments, a therapeutically effective amount treats or prevents a disease or a disorder, e.g., ameliorates at least one sign or symptom of the disorder. In various embodiments, the disease or disorder is a cancer.

The terms “optional” and “optionally” as used herein mean that the subsequently described event or circumstance may or may not occur, and that the description includes instances where the event or circumstance occurs and instances in which it does not. For example, “optionally substituted aryl” encompasses both “aryl” and “substituted aryl” as defined herein.

The terms “alkylene” and “alkylenyl” as used herein refers to a divalent analog of a linear or branched alkyl group. Non-limiting examples of alkylene groups include —CH₂—, —CH₂CH₂—, —CH₂CH₂CH₂—, —(CH₂)₄—, —(CH₂)₅—, and —(CH₂)₆—.

The term “aryl” as used herein refers to a monocyclic aromatic hydrocarbon group or a multicyclic group that contains at least one aromatic hydrocarbon ring. In certain embodiments, an aryl group has from 6 to 15 or more, or 6 to 12 or more, or 6 to 10 or more, ring atoms. Non-limiting examples of aryl groups include phenyl, naphthyl, fluorenyl, azulenyl, anthryl, phenanthryl, biphenyl and terphenyl. The aromatic hydrocarbon ring of an aryl group may be attached or fused to one or more saturated, partially unsaturated, or aromatic rings—e.g., dihydronaphthyl, indenyl, indanyl and tetrahydronaphthyl (tetralinyl). An aryl group may optionally be substituted with one or more substituents as described herein.

The term “cycloalkyl” as used herein refers to a saturated or unsaturated monocyclic, bicyclic, other multicyclic, or bridged cyclic hydrocarbon group. A cycloalkyl group can have 3-22, 3-12, or 3-8 ring carbons, referred to herein as (C₃-C₂₂)cycloalkyl, (C₃-C₁₂)cycloalkyl, or (C₃-C₈)cycloalkyl, respectively. A cycloalkyl group can also have one or more carbon-carbon double bond or carbon-carbon triple bond.

The term “heterocyclyl” refers to cyclic groups containing at least one heteroatom as a ring atom, in some cases, 1 to 3 heteroatoms as ring atoms, with the remainder of the ring atoms being carbon atoms. Suitable heteroatoms include oxygen, sulfur, nitrogen, phosphorus, and the like. In some cases, the heterocycle may be 3- to 10-membered ring structures or 3- to 7-membered rings, whose ring structures include one to four heteroatoms.

The term “heteroaryl” as used herein refers to a mono-, bi-, or multi-cyclic aromatic ring system containing one or more heteroatoms, for example 1-3 heteroatoms, such as nitrogen, oxygen, and sulfur. Heteroaryls can also be fused to non-aromatic rings. In various embodiments, the term “heteroaryl” as used herein represents a stable 5- to 7-membered monocyclic, stable 9- to 10-membered fused bicyclic, or stable 12- to 14-membered fused tricyclic heterocyclic ring system which contains an aromatic ring that contains at least one heteroatom selected from the group consisting of N, O, and S. In some embodiments, at least one nitrogen is in the aromatic ring.

The term “perfluoroaryl” used herein refers to an aryl group where every hydrogen atom is replaced with a fluorine atom. In some embodiments, the perfluoroaryl is 2,3,5,6-tetrafluorophenylene or 2,2′,3,3′,5,5′,6,6′-octafluoro-1,1′-biphenyl-4,4′-ene.

All numerical ranges herein include all numerical values and ranges of all numerical values within the recited range of numerical values. As a non-limiting example, (C₁-C₆) alkyls also include any one of C₁, C₂, C₃, C₄, C₅, C₆, (C₁-C₂), (C₁-C₃), (C₁-C₄), (C₁-C₅), (C₂-C₃), (C₂-C₄), (C₂-C₅), (C₂-C₆), (C₃-C₄), (C₃-C₅), (C₃-C₆), (C₄-C₅), (C₄-C₆), and (C₅-C₆) alkyls.

The term “pharmaceutically acceptable salt(s)” used herein refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

The term “pharmaceutically acceptable carrier” used herein refers to a pharmaceutically-acceptable material, composition or vehicle, such as a liquid or solid filler, diluent, excipient, solvent or encapsulating material, involved in carrying or transporting any supplement or composition, or component thereof, from one organ, or portion of the body, to another organ, or portion of the body. Each carrier must be “acceptable” in the sense of being compatible with the other ingredients of the formulation and not injurious to the patient.

As used herein, the term “linker” refers to a carbon chain that can contain heteroatoms (e.g., nitrogen, oxygen, sulfur, etc.) and which may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 atoms long. Linkers may be substituted with various substituents disclosed herein. Examples of linkers include, but are not limited to, pH-sensitive linkers, protease cleavable peptide linkers, nuclease sensitive nucleic acid linkers, lipase sensitive lipid linkers, glycosidase sensitive carbohydrate linkers, hypoxia sensitive linkers, photo-cleavable linkers, heat-labile linkers, enzyme cleavable linkers (e.g., esterase cleavable linker), ultrasound-sensitive linkers, x-ray cleavable linkers, and so forth.

Each group described herein (including without limitation alkyl, alkylene, cycloalkyl, heterocyclyl, aryl, heteroaryl), whether as a primary group or as a substituent group, may optionally be substituted with one or more substituents, in certain embodiments with one to six or more substituents, independently selected from the group consisting of halide, cyano, nitro, hydroxyl, sulfhydryl, amino, —OR₈, —SR₈, —NR₉R₁₀, alkyl, alkenyl, alkynyl, haloalkyl, hydroxyalkyl, aminoalkyl, -alkylcycloalkyl, -alkylheterocyclyl, -alkylaryl, -alkylheteroaryl, cycloalkyl, heterocyclyl, aryl, heteroaryl, —C(═O)R₈, —C(NR₁₁)R₈, —S(═O)R₈, —S(═O)₂R₈, —S(═O)₂OR₁₂, —C(═O)OR₁₂, —OC(═O)R₈, —C(═O)NR₉R₁₀, —NR₈C(═O)R₈, —S(═O)₂NR₉R₁₀, —NR₈S(═O)₂R₈, —OC(═O)OR₈, —OC(═O)NR₉R₁₀, —NR₈C(═O)OR₈, —NR₈C(═O)NR₉R₁₀, —NR₈C(═NR¹¹)NR₉R₁₀, —P(═O)(R₈)₂, —P(═O)(OR₁₂)R₈, —P(═O)(OR₁₂)₂, —OP(═O)(R₈)₂, —OP(═O)(OR₁₂)R₈, and —OP(═O)(OR₁₂)₂, wherein: each occurrence of R₈ independently is hydrogen, alkyl, haloalkyl, hydroxyalkyl, aminoalkyl, -alkylcycloalkyl, -alkylheterocyclyl, -alkylaryl, -alkylheteroaryl, cycloalkyl, heterocyclyl, aryl or heteroaryl; each occurrence of R₉ and R₁₀ independently is hydrogen, alkyl, haloalkyl, hydroxyalkyl, aminoalkyl, -alkylcycloalkyl, -alkylheterocyclyl, -alkylaryl, -alkylheteroaryl, cycloalkyl, heterocyclyl, aryl or heteroaryl, or R₉ and R₁₀, together with the nitrogen atom to which they are attached, form a heterocyclic or heteroaryl ring; each occurrence of R₁₁ independently is hydrogen, —OR₈, alkyl, haloalkyl, hydroxyalkyl, aminoalkyl, -alkylcycloalkyl, -alkylheterocyclyl, -alkylaryl, -alkylheteroaryl, cycloalkyl, heterocyclyl, aryl or heteroaryl; and each occurrence of R₁₂ independently is hydrogen, alkyl, haloalkyl, hydroxyalkyl, aminoalkyl, -alkylcycloalkyl, -alkylheterocyclyl, -alkylaryl, -alkylheteroaryl, cycloalkyl, heterocyclyl, aryl or heteroaryl.

### General Overview of the Invention

The present disclosure provides platinum(IV) prodrug—perfluoroaryl peptide conjugates, pharmaceutical compositions, methods of producing such compositions and methods of using the same. The platinum(IV) prodrug can be reduced to platinum(II) complexes without an axial ligand by a redox-active molecule inside tumor/cancer cells, and can overcome any tumor/cancer resistance to platin compounds, e.g., by being converted to antitumor-active platinum(II) complexes intracellularly. One advantage of the present disclosure is that by conjugating perfluoroaryl peptide with platinum(IV) prodrug complex, the achieved platinum perfluoroaryl peptide conjugates can increase the in vivo stability of the platinum prodrug and increases the distribution of platinum to the target cell, e.g. cancer cells. This can result in better efficiency and tolerability, making the conjugates suitable for potential clinical development.

In a first aspect, the disclosure provides a conjugate of a platinum(IV) prodrug covalently associated with a perfluoroaryl peptide. In some embodiments, the platinum(IV) prodrug is covalently directly linked to the perfluoroaryl peptide. In some embodiments, the platinum(IV) prodrug is covalently associated with the perfluoroaryl peptide by a linker.

In some embodiments, the conjugate has the formula: C2-L2-Pt-L1-C1, wherein Pt is a platinum(IV) prodrug; L1, L2 are absent or a linker; C1 is a first perfluoroaryl peptide; C2 is absent or a second perfluoroaryl peptide, wherein the first perfluroaryl peptide may be identical to or different from the second perfluoroaryl peptide. L1 and L2 might be the same or different linkers. In some embodiments, the conjugate has the formula: Pt-L1-C1. In some embodiments, the peptide comprises two cysteines. In some embodiments, the two cysteines connect the perfluoroaryl to form a marcocyclic ring. In another embodiment, the peptide comprises two lysines. In another embodiment, the two lysines connect the perfluoraryl to form a macrocyclic ring.

In some embodiments, the conjugate further comprises another moiety that facilitates delivery of Pt(IV) prodrug across the blood brain barrier. In some embodiments, the moiety is insulin, transferrin, insulin like growth factor (IGF), leptin, low density lipoprotein (LDL), or fragments or peptidomimetics or derivatives thereof.

In some embodiments, the peptide comprises the amino acid sequence of A-G-Y-L-L-G-K-I-N-L-K-A-C-A-A-L-A-K-K-C-L (SEQ ID NO:1), A-C-Y-L-L-G-K-I-C-L-K-A-L-A-A-L-A-K-K-I-L (SEQ ID NO:3), or A-G-Y-L-L-G-K-I-N-L-K-A-L-C-A-L-A-K-K-I-C(SEQ ID NO:4).

In some embodiments, the platinum(IV) prodrug is any platinum(IV) complex. In some embodiments, the platinum(IV) prodrug is derived from oxidizing Pt(II) complex with the addition of two axial ligands.

In some embodiments, the platinum(IV) prodrug is derived from oxidizing cisplatin, carboplatin, or oxaliplatin with the addition of two axial ligands. Bioactive functional groups can be appended to the Pt(IV) center through the axial ligands. In some embodiments, the Pt(IV) prodrug is functionalized symmetrically with ligands containing carboxylates. These can be used to attach peptides to the Pt(IV) prodrugs that improve cellular uptake or target the drug to a particular cell type. In some embodiments, the Pt(IV) prodrug is functionalized asymmetrically. In some embodiments, the Pt(IV) prodrug is associated with two perfluoroaryl peptides via the two axial ligands. In some embodiments, the two perfluoroaryl peptides are the same. In another embodiment, the two perfluoroaryl peptides are different. In certain embodiments, peptides that can be used to improve cellular uptake include multivalent and/or multispecific binding proteins that specifically bind to receptors expressed on the brain vascular epithelium. In some embodiments, the receptors expressed on the brain vascular epithelium include insulin receptor, transferrin receptor, LRP, melanocortin receptor, nicotinic acetylcholine receptor, VACM-1 receptor, vascular endothelial growth factor receptors 1, 2 and 3, glucocorticoid receptor, ionotropic glutamate receptor, M3 receptor, aryl hydrocarbon receptor, GLUT-1, inositol-1,4,5-trisphosphate (IP3) receptor, N-methyl-D-aspartate receptor, S1P1, P2Y receptor, and RAGE.

In some embodiments, peptides that can be used to improve cellular uptake included antibodies. The term “antibody” refers to an immunoglobulin (Ig) molecule, which is generally comprised of four polypeptide chains, two heavy (H) chains and two light (L) chains, or a functional fragment, mutant, variant, or derivative thereof, that retains the epitope binding features of an Ig molecule. Such fragment, mutant, variant, or derivative antibody formats are known in the art. In an embodiment of a full-length antibody, each heavy chain is comprised of a heavy chain variable region (VH) and a heavy chain constant region (CH). The CH is comprised of three domains, CH1, CH2 and CH3. Each light chain is comprised of a light chain variable region (VL) and a light chain constant region (CL). The CL is comprised of a single CL domain. The VH and VL can be further subdivided into regions of hypervariability, termed complementarity determining regions (CDRs), interspersed with regions that are more conserved, termed framework regions (FRs). Generally, each VH and VL is composed of three CDRs and four FRs, arranged from amino-terminus to carboxy-terminus in the following order: FR1, CDR1, FR2, CDR2, FR3, CDR3, and FR4. Immunoglobulin molecules can be of any type (e.g., IgG, IgE, IgM, IgD, IgA and IgY), class (e.g., IgG1, IgG2, IgG3, IgG4, IgA1 and IgA2), or subclass.

In some embodiments, the Pt(IV) prodrug comprises two axial ligands, and each ligand is independently selected from the group consisting of —O-alkyl, —O-alkyl-C(═O)O-alkyl, —O-alkyl-C(═O)OH, —OC(═O)-alkyl, —OC(═O)-alkyl-C(═O)O-alkyl, —OC(═O)-alkyl-C(═O)OH, —OC(═O)NH-alkyl, —OC(═O)NH-alkyl-C(═O)O-alkyl, and —OC(═O)NH-alkyl-C(═O)OH wherein each occurrence of alkyl independently contains 2 to 15 carbon atoms, is straight-chain or branched, and is optionally substituted.

In some embodiments, the platinum(IV) prodrug may comprise at least one ligand which functions as a linker, and is selected such that the perfluoroaryl peptide is linked to the Pt(IV) prodrug. In some embodiments, the platinum is connected to a perfluoroaryl peptide at one axial position and has a —OC(═O)R₂ or —OC(═O)NHR₂ ligand at the other axial position, wherein R₂ is C₁-C₁₀ alkyl.

In some embodiments, the platinum(IV) prodrug is selected from the group consisting of

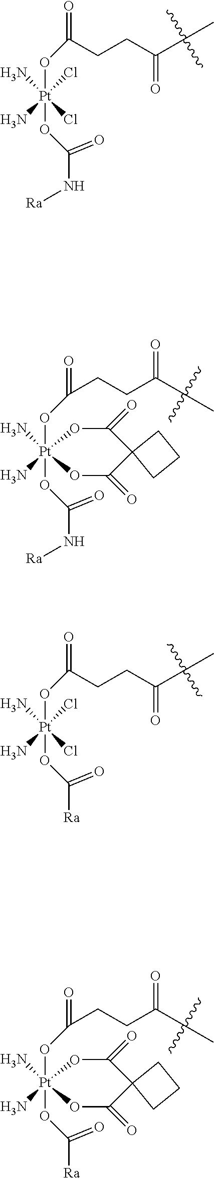

Ra is C₁-C₁₀ alkyl; and an atom adjacent to a wavy line is a site where the platinum (IV) prodrug is connected to the reminder of the conjugate. In some embodiments, Ra is C₃-C₆ alkyl.

In some embodiments, the perfluoroaryl peptide comprises a structure represented by a formula selected from the group consisting of:

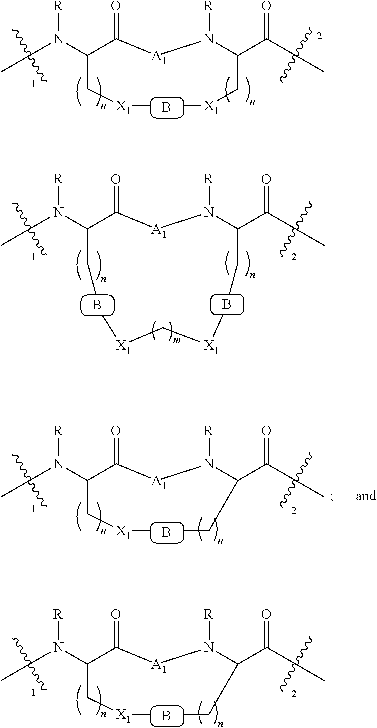

wherein

A₁ comprises 1-20 natural or unnatural amino acids;

X₁ is S or NH;

R is H or alkyl;

n is 0, 1, 2, 3, 4, 5, or 6;

m is 1, 2, 3, 4, 5, or 6; and

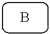

is a perfluoroaryl para-substituted diradical.

The perfluoroaryl peptide may be connected with the platinum(IV) prodrug via wavy line 1 or wavy line 2. In some embodiments, the atom adjacent to wavy line 1 is further connected with a plurality of amino acids, and the platinum(IV) prodrug is connected with the perfluoroaryl peptide via wavy line 1.

In some embodiments,

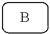

is 2,3,5,6-tetrafluorophenylene or 2,2′,3,3′,5,5′,6,6′-octafluoro-1,1′-biphenyl-4,4′-ene. In some embodiments, R is H.

In some embodiment, the perfluoroaryl peptide is represented by the following formula:

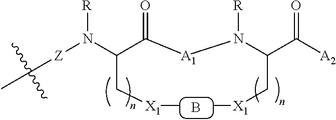

wherein
A₁ comprises 1-20 natural or unnatural amino acids;
A₂ is OH, NH₂, a carboxylate protecting group, a natural or unnatural amino acid, a peptide, an oligopeptide, a polypeptide, or a protein;
R is H or alkyl;
X₁ is S or NH;
n is 0, 1, 2, 3, 4, 5, or 6;
Z is a natural or unnatural amino acid, a plurality of natural amino acids or unnatural amino acids; a peptide, an oligopeptide, a polypeptide, and

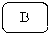

is a perfluoroaryl para-substituted diradical.

In some embodiments, none of Z and A₁ comprises cysteine. In some embodiments, one or both of Z and A₁ comprises arginine, histidine, lysine, aspartic acid, glutamic acid, serine, threonine, asparagine, glutamine, glycine, praline, alanine, valine, isoleucine, leucine, methionine, phenylalanine, tyrosine, or tryptophan.

In some embodiments, the platinum(IV) prodrug perfluororaryl peptide conjugate is represented by the following formula:

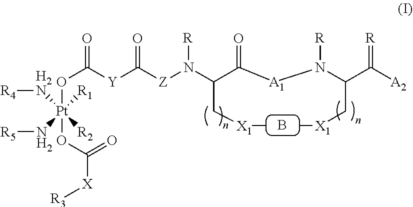

or a pharmaceutically acceptable salt thereof, wherein
R₁ and R₂ each is Cl, or R₁ and R₂ are joined to form an oxalate or

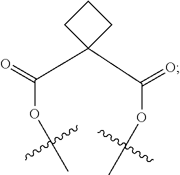 [SMILES: *OC(=O)C1(C(=O)O*)CCC1]

R₃ is selected from the group consisting of alkyl, cycloalkyl, heterocyclyl, aryl, and heteroaryl, wherein each of the alkyl, cycloalkyl, heterocyclyl, aryl, and heteroaryl groups optionally is substituted with one or more groups;
R₄ and R₅ are each H or together constitute a cyclohexyl ring;
R is H or alkyl;
X is absent or NH;
X₁ is S or NH;
Y is selected from the group consisting of alkylene and aryl;
Z is absent, a natural or unnatural amino acid, a plurality of natural amino acids or unnatural amino acids, a peptide, an oligopeptide, a polypeptide, or a protein;
A₁ comprises 1-20 natural or unnatural amino acids;
A₂ is OH, NH₂, a carboxylate protecting group, a natural or unnatural amino acid, a peptide, an oligopeptide, a polypeptide, or a protein;
n is 0, 1, 2, 3, 4, 5, or 6; and

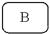

is a perfluoroaryl para-substituted diradical.

In some embodiments, Y is C₁-C₆ alkylene. In some embodiment, n is 1. In some embodiments,

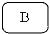

is 2,3,5,6-tetrafluorophenylene or 2,2′,3,3′,5,5′,6,6′-octafluoro-1,1′-biphenyl-4,4′-ene.

In some embodiments, the conjugate is selected from the group consisting of (SEQ ID NO: 5):

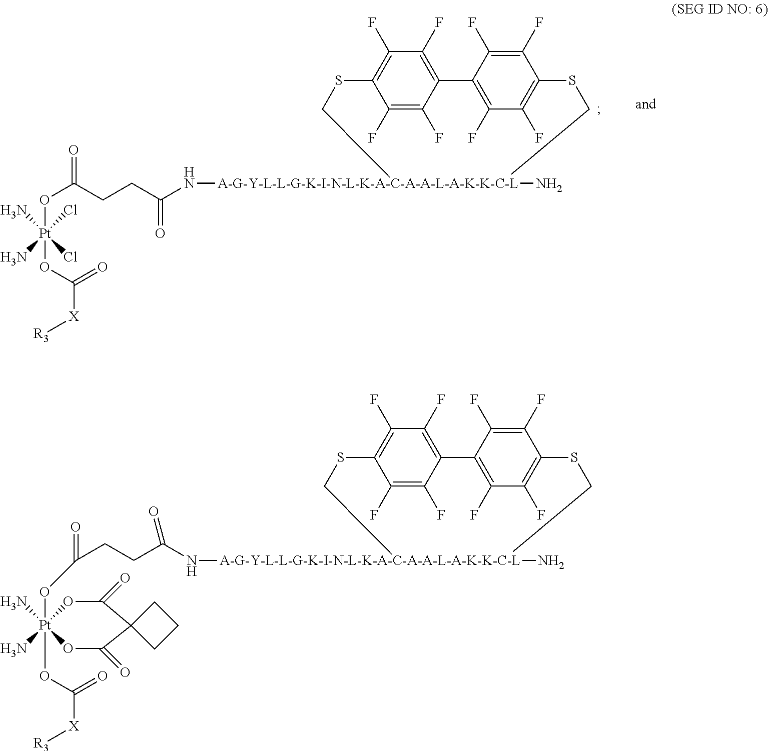

wherein X is NH or absent;

R₃ is selected from the group consisting of alkyl, cycloalkyl, heterocyclyl, aryl, and heteroaryl, wherein each of the alkyl, cycloalkyl, heterocyclyl, aryl, and heteroaryl groups optionally is substituted. In some embodiments, R₃ is C₂-C₆ alkyl.

In some embodiments, the conjugate is represented by the formula as follows (SEQ ID NO: 7):

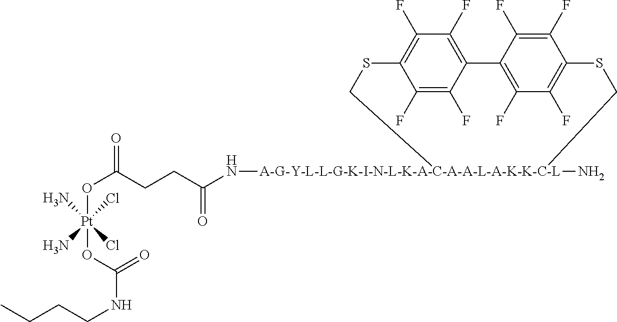

and pharmaceutically acceptable salts thereof.

In another embodiment, the conjugate is represented by the formula as follows, wherein each of the two amino acid sequences bound to the cisplatin group are represented by (SEQ ID NO: 8):

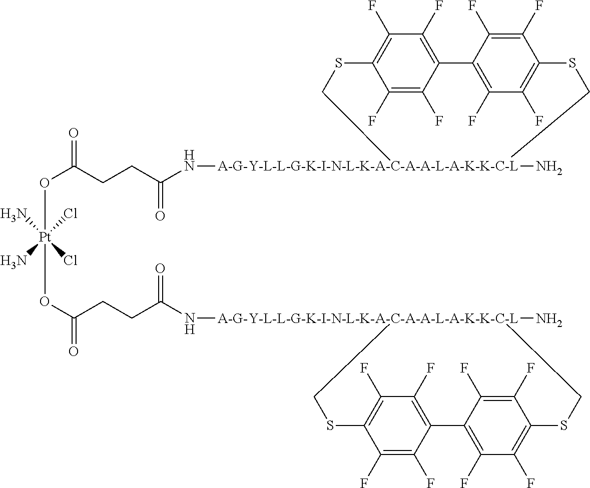

In a second aspect, the preset disclosure provides methods of preparing the conjugates disclosed herein. In general, the conjugates of the present disclosure may be prepared by the methods illustrated in the general reaction schema described below, or by modifications thereof, using readily available starting materials, reagents and conventional synthesis procedures. In these reactions, it is also possible to make use of variants which are in themselves known, but are not mentioned here.

Referring to schemes 1 and 2, a conjugate of the present disclosure may, in some embodiments, be prepared by:

a) providing a platinum prodrug with a functional group by (see Scheme 1):
A. oxidizing Pt(II) compound with hydrogen peroxide in water to obtain dihydroxy Pt(IV) complex;
B. reacting the dihydroxy Pt(IV) complex with succinic anhydride in dimethylformamide to generate asymmetric monosuccinate Pt complex, as well as the symmetric disuccinate Pt(IV) complex; from the reaction mixture, symmetric disuccinate is removed by acetone as the disuccinate is soluble in acetone and the asymmetric monosuccinate is not.
C. reacting the asymmetric monosuccinate Pt(IV) complex with n-butyl isocyanate to afford the final asymmetric Pt(IV) prodrug;

b) providing a peptide comprising two cysteines; the peptide is prepared by automated fast-flow peptide synthesis;

c) reacting the platinum(IV) prodrug from step a) with the peptide from step b) to form a platinum(IV) prodrug peptide conjugate; the Pt(IV) prodrug is coupled to the N-terminus of each resin-bound peptide using standard active ester chemistry; the prodrug-peptide conjugates are cleaved from the resin with trifluoroacetic acid and purified by reversed-phase high-performance liquid chromatography (RP-HPLC);

d) reacting the platinum prodrug peptide with perfluoroaryl to form a platinum(IV) prodrug perfluroaryl peptide conjugate; the cysteine residues of the prodrug-peptide conjugate were cyclized using perfluroaryl (see Scheme 2).

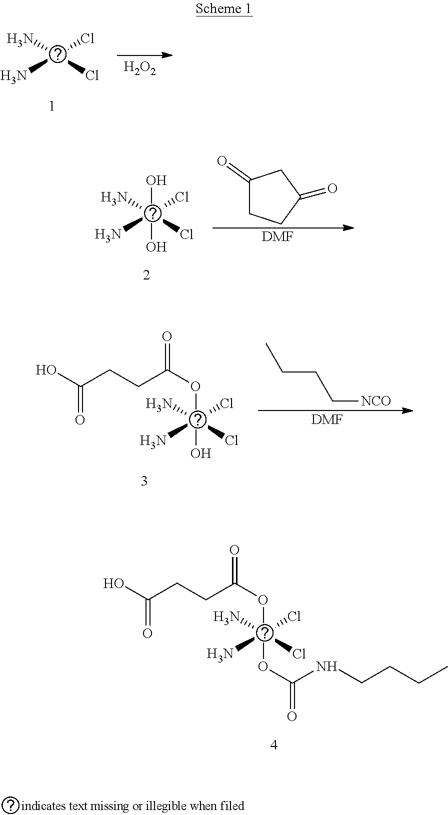

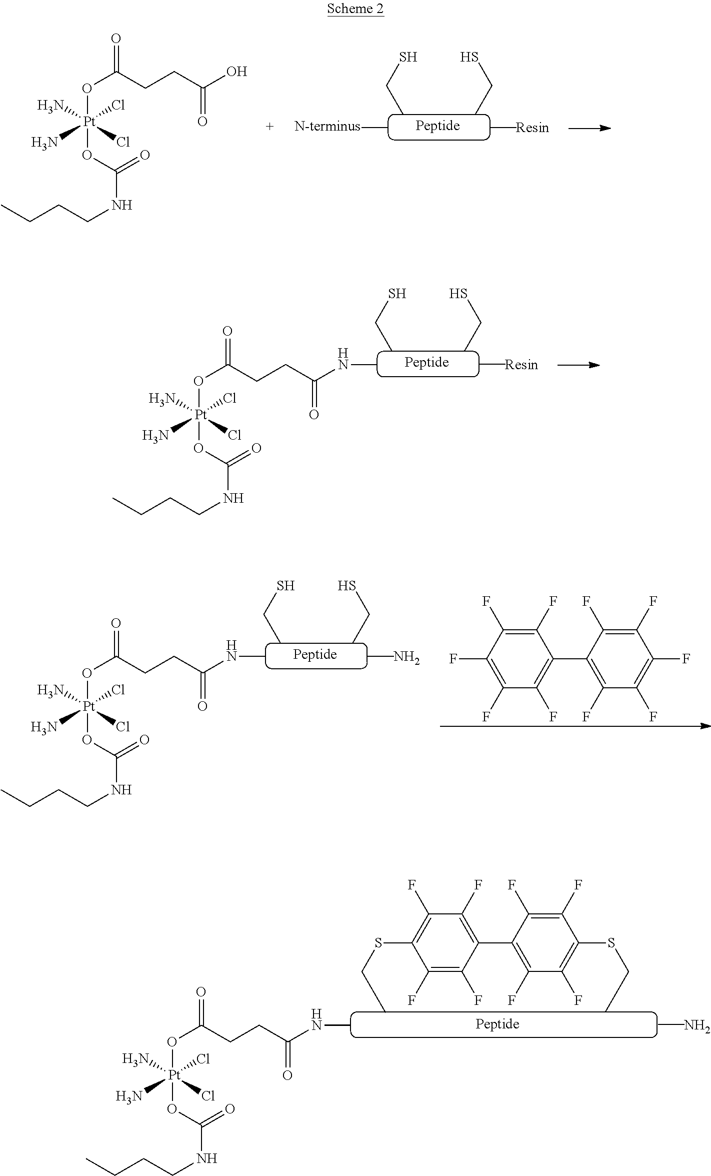

In some embodiments, the conjugates of the present disclosure may be prepared by: a) reacting a peptide with a linker to form a linker-peptide; b) reacting a platinum(IV) prodrug with the linker-peptide to form a platinum(IV) prodrug peptide conjugate wherein the platinum prodrug and the peptide are linked via the linker; c) reacting the platinum prodrug peptide with perfluoroaryl to form a platinum(IV) prodrug perfluoroaryl peptide conjugate, wherein the platinum(IV) prodrug is associated with the perfluoroaryl peptide via the linker.

In some embodiments, the present disclosure also provides methods of preparing the Pt(IV) prodrug perfluoroaryl peptide conjugates disclosed herein according to Scheme 3.

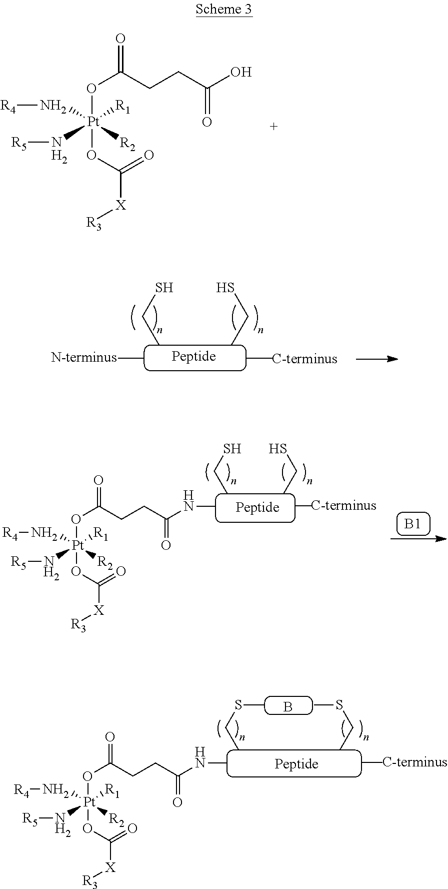

wherein
R₁ and R₂ each is Cl, or R₁ and R₂ are joined to form an oxalate or

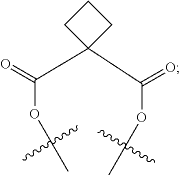 [SMILES: *OC(=O)C1(C(=O)O*)CCC1]

R₃ is selected from the group consisting of alkyl, cycloalkyl, heterocyclyl, aryl, and heteroaryl, wherein each of the alkyl, cycloalkyl, heterocyclyl, aryl, and heteroaryl groups optionally is substituted with one or more groups;
R₄ and R₅ are each H or together constitute a cyclohexyl ring;
n is 0, 1, 2, 3, 4, 5, or 6;
X is absent or NH;

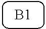

is a perfluoroaryl compound;

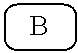

is a perfluoroaryl para-substituted diradical.

In some embodiments,

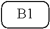

is hexafluorophenyl or decafluorobiphenyl; and

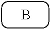

is 2,3,5,6-tetrafluorophenylene or 2,2′,3,3′,5,5′,6,6′-octafluoro-1,1′-biphenyl-4,4′-ene.

In some embodiments, the present disclosure also provides methods of preparing the Pt(IV) prodrug perfluoroaryl peptide conjugates disclosed herein according to Scheme 4.

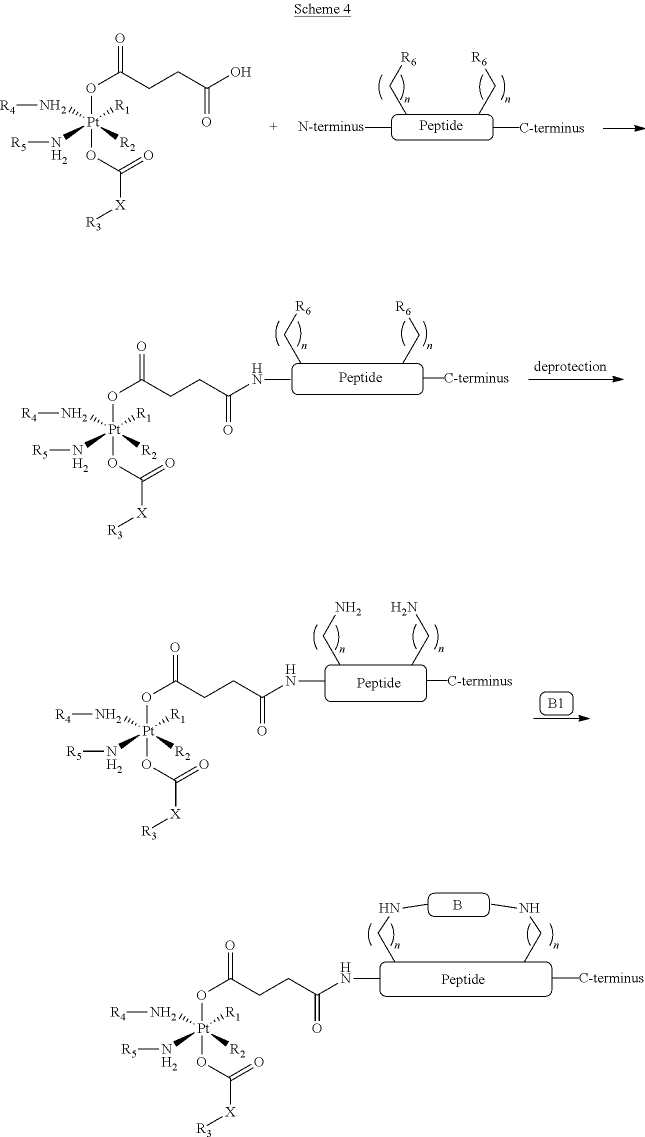

wherein
R₁ and R₂ each is Cl, or R₁ and R₂ are joined to form an oxalate or

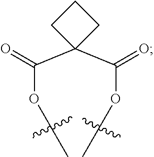 [SMILES: *OC(=O)C1(C(=O)O*)CCC1]

R₃ is selected from the group consisting of alkyl, cycloalkyl, heterocyclyl, aryl, and heteroaryl, wherein each of the alkyl, cycloalkyl, heterocyclyl, aryl, and heteroaryl groups optionally is substituted with one or more groups;
R₄ and R₅ are each H or together constitute a cyclohexyl ring;
R₆ is a protected amino group;
n is 0, 1, 2, 3, 4, 5, or 6;
X is absent or NH;

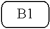

is a perfluoroaryl compound;

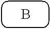

is a perfluoroaryl para-substituted diradical.

In some embodiments the peptide comprises two lysines. Any of a variety of methods can be used to associate a platinum(IV) prodrug with a perfluoroaryl peptide.

In a third aspect, the present disclosure provides pharmaceutical compositions comprising a Pt(IV)—perfluoroaryl peptide conjugate, as described herein, and optionally a pharmaceutically acceptable carrier. In some embodiments, the Pt(IV)—perfluoroaryl peptide conjugates are provided in an effective amount in the pharmaceutical composition. In some embodiments, the effective amount is a therapeutically effective amount. In some embodiments, the effective amount is a prophylactically effective amount.

Pharmaceutical compositions described herein can be prepared by any method known in the art of pharmacology. In general, such preparatory methods include the steps of bringing the Pt(IV)—perfluoroaryl peptide conjugates into association with a carrier and/or one or more other accessory ingredients, and then, if necessary and/or desirable, shaping and/or packaging the product into a desired single- or multi-dose unit.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

In a fourth aspect, the present disclosure provides methods for treating a disease or condition. The method comprises administering a therapeutically effective amount of the Pt(IV) prodrug—perfluoroaryl peptide conjugates and a pharmaceutical composition thereof to a subject in need thereof. In one embodiment, the disease is a cancer or proliferative disease, for example, lymphoma, renal cell carcinoma, leukemia, prostate cancer, lung cancer, pancreatic cancer, melanoma, colorectal cancer, ovarian cancer, breast cancer, glioblastoma multiforme and leptomeningeal carcinomatosis. In one embodiment, the disease is glioblastoma.

In some embodiments, the Pt(IV) prodrug—perfluoroaryl peptide conjugates disclosed herein may assist in delivering Pt to the site of a tumor, e.g., the concentration of Pt at a tumor site, after administration Pt(IV) prodrug—perfluoroaryl peptide conjugates to a subject that has a tumor, is higher than administration Pt(II) drug not associated with perfluoroaryl peptides. Without attempting to limit the scope of the present disclosure, the accumulation may be due, at least in part, the presence of perfluoroaryl peptide, which allows Pt(IV) prodrug to reach specifically at a tumor site. In one embodiment, the tumor is brain cancer.

### Administration

The Pt(IV) prodrug—perfluoroaryl peptide conjugates disclosed herein may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way which allows them across the blood-brain barrier, vascular barrier, or other epithelial barrier.

### Dosing

The present disclosure provides methods comprising administering the Pt(IV) prodrug—perfluoroaryl peptide conjugates to a subject in need thereof. The Pt(IV) prodrug—perfluoroaryl peptide conjugates as described herein may be administered to a subject using any amount and any route of administration effective for preventing or treating or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like.

Compositions in accordance with the present disclosure are typically formulated in dosage unit form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, compositions in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

As used herein, a “split dose” is the division of single unit dose or total daily dose into two or more doses, e.g, two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose. In some embodiments, the Pt(IV) prodrug—perfluoroaryl conjugates of the present disclosure are administered to a subject in split doses. The Pt(IV) prodrug—perfluoroaryl conjugates may be formulated in buffer only or in a formulation described herein.

### EXAMPLES

### Example 1: Synthesis of Pt(IV) Prodrug

Pt(IV) prodrug synthesis began with the oxidation of cisplatin (1) to 2 with hydrogen peroxide in water (Scheme 1). Reaction with succinic anhydride in dimethylformamide allowed the generation of asymmetric Pt complex 3, as well as the symmetric disuccinate. From the reaction mixture, symmetric disuccinate is removed by acetone as the disuccinate is soluble in acetone and the asymmetric monosuccinate is not. Finally, complex 3 was reacted with n-butyl isocyanate to afford the final asymmetric Pt(IV) prodrug 4, which has a single carboxylate for subsequent functionalization. The single carboxylate can be used for attachment to a peptide on resin. Pt(IV) prodrug 4 was characterized by 1H-NMR spectroscopy, electrospray ionization mass spectrometry, and elemental analysis.

### Example 2: Synthesis of Prodrug-Peptide Conjugate

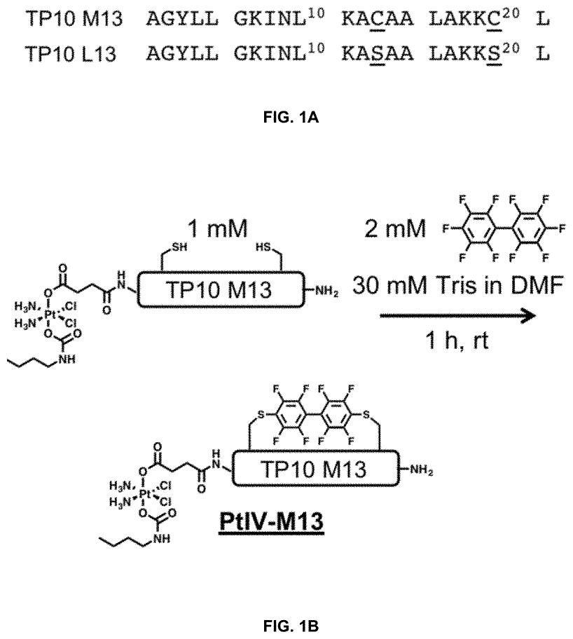

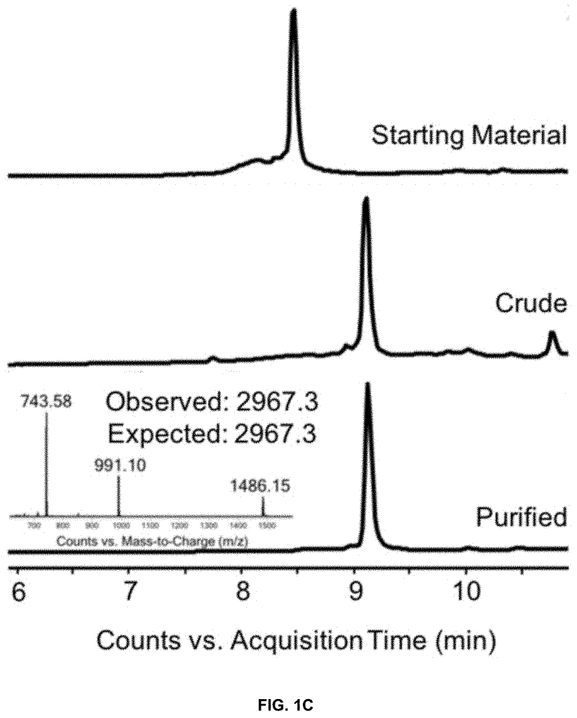

TP10 M13 (AGYLLGKINLKACAALAKKCL; SEQ ID NO: 1) containing two cysteine residues for cyclization was prepared by automated fast-flow peptide synthesis. Additionally, a linear control sequence (TP10 L13; AGYLLGKINLKASAALAKKSL; SEQ ID NO:2) was synthesized in which serine residues were substituted for cysteine (FIG. 1A). Prodrug 4 was coupled to the N-terminus of each resin-bound peptide using standard active ester chemistry. The prodrug-peptide conjugates were cleaved from the resin with trifluoroacetic acid and purified by reversed-phase high-performance liquid chromatography (RP-HPLC). Next, the cysteine residues of the prodrug-peptide conjugate were cyclized using decafluorobiphenyl. The product (PtIV-M13) was characterized by liquid chromatography-mass spectrometry (LC-MS) (FIG. 1C). No reduction of the prodrug was observed during the perfluoroaryl cyclization reaction, despite the presence of two unprotected thiols.

### Example 3: Cytotoxicity Evaluation

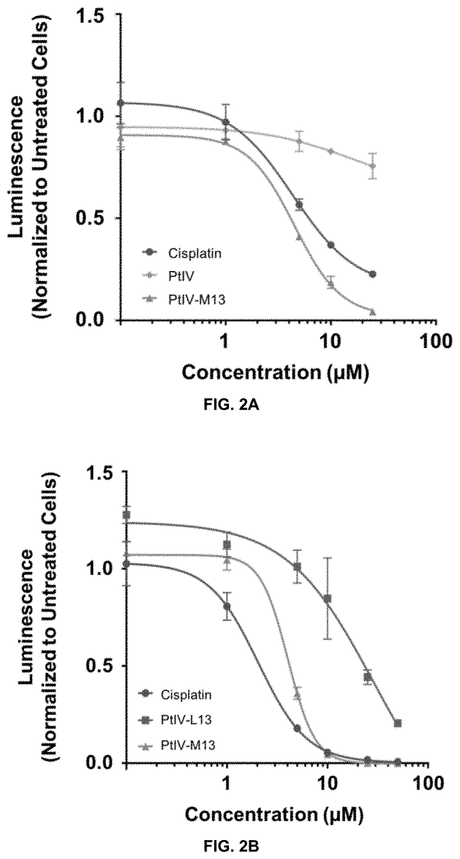

The cytotoxicity of the PtIV-M13 conjugate was evaluated against patient-derived glioma cells (GCs). To evaluate the in vitro efficacy of the conjugate, G9 neurospheres were treated with PtIV-M13, prodrug 4, and cisplatin for 80 hours. After treatment, the potency of the compounds was assessed by the CellTiter-Glo luminescent cell viability assay (FIGS. 2A and 2B). The assay involves lysis of the cells and quantitation of the amount of ATP present, which is directly proportional to the number of living cells in culture. The IC₅₀ of PtIV-M13 conjugate against the G9 cells was determined to be 4.51±0.22 μM, which is similar to cisplatin at 4.34±0.66 μM. However, prodrug 4 was not potent, perhaps due to an inability to enter cells (FIG. 2A). As a result, in the follow-up in vitro experiment with another GC line, PtIV-L13 was used as a control instead as this should have similar properties to the PtIV-M13, only without the perfluoroaryl. The same experiment was repeated on G30 GCs. The PtIV-M13 conjugate had similar potency of 4.04 μM. The linear control conjugate PtIV-L13 was not as potent, with an IC₅₀ of 28.75 μM, suggesting that the perfluoroaryl macrocycle helps improve the potency of the prodrug-peptide conjugate (FIG. 2B).

### Example 4: Cellular Uptake and Cellular Localization of PtIV-M13

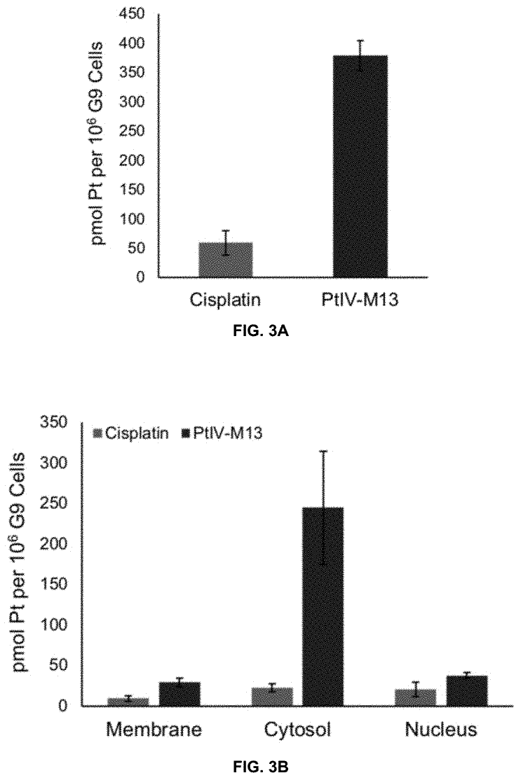

G9 GSCs were treated with 5 μM of cisplatin or the PtIV-M13 conjugate for 5 hours. The whole cell concentration of platinum was then evaluated by graphite furnace atomic absorption spectroscopy (GFAAS). The amount of platinum in the cells after treatment with the PtIV-M13 conjugate was six times greater than with cisplatin, suggesting the PtIV-M13 conjugate has superior uptake into cells (FIG. 3A). The subcellular distribution was examined by fractionating the different cellular components. Most of the platinum from PtIV-M13 is present in the cytosol. The amount of nuclear platinum is relatively similar between the PtIV-M13 conjugate and cisplatin (FIG. 3B).

### Example 5: In Vivo Evaluation of PtIV-M13

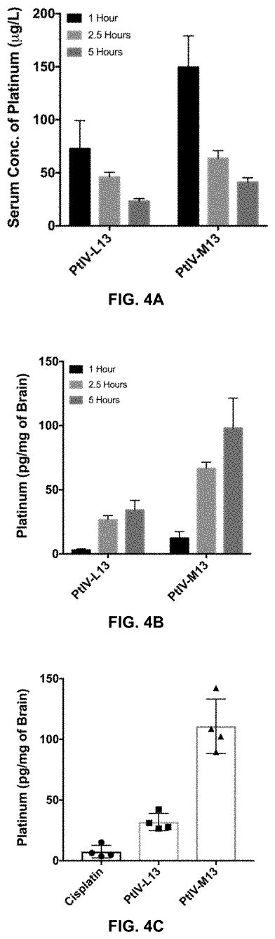

Pharmacokinetic and biodistribution studies were performed in healthy mice. For pharmacokinetics, 100 μL of a 100 μM solution of PtIV-M13 or PtIV-L13 dissolved in saline (corresponding to a dose of ˜2.5 mg/kg) were injected into the tail veins of mice. The brain and a blood sample were isolated at 1, 2.5, and 5 hours. A portion of the brain was dissolved in nitric acid and the amount of platinum in the brain and serum were quantified by GFAAS. For both PtIV-M13 and PtIV-L13, the serum concentration decreased over time and the brain distribution increased over time (FIGS. 4A and 4B). FIG. 4A shows the concentration of platinum in serum at 1, 2.5, and 5 hours after treatment with either PtIV-M13 or the linear control PtIV-L13 (n=3 animals for each time point). The error bars are the standard deviation from three different animals injected with the compound. FIG. 4B shows the brain concentration of platinum at 1, 2.5, and 5 hours after treatment with either PtIV-M13 or the linear control PtIV-L13 (n=3 animals for each time point). The error bars are the standard deviation from three different animals injected with the compound. Although the read-out is platinum, platinum(II) is rapidly cleared by the kidney, suggesting that the majority of the quantified platinum in the serum comes from intact conjugates. Therefore, the results indicate that PtIV-M13 is more stable in serum and has more brain uptake than its linear control.

To study the biodistribution, 100 μL of a 100 μM solution of PtIV-M13, PtIV-L13, or cisplatin dissolved in normal saline were injected into the tail vein of mice. After five hours, the mice were sacrificed and the brain, heart, lungs, kidney, spleen and liver were removed. The amount of platinum in each organ was measured by dissolving a portion of the respective organ in nitric acid and then quantifying the amount of platinum by GFAAS (supporting information). The brains of the mice treated with the PtIV-M13 conjugate exhibited a 15-fold increase in the amount of platinum compared with cisplatin, suggesting the perfluoroaryl macrocycle improves the amount of drug that reaches the brain (FIG. 4C). FIG. 4C shows the concentration of platinum in the brain of mice 5 hours after treatment with either PtIV-M13, PtIV-L13, or cisplatin (n=4 animals for each treatment group). The results are significant with a p-value of less than 0.0001 by a One-Way ANOVA test.

In conclusion, covalent attachment of a Pt(IV) prodrug to a brain penetrant perfluoroaryl macrocyclic peptide increases the in vivo stability of the prodrug and increases the distribution of platinum to the brain. The PtIV-M13 conjugate is the first compound to combine the benefits of oxidized platinum prodrugs with perfluoroaryl macrocyclic peptides.

### REFERENCES

(1) Stupp, R.; Mason, W. P.; van den Bent, M. J.; Weller, M.; Fisher, B.; Taphoorn, M. J. B.; Belanger, K.; Brandes, A. A.; Marosi, C.; Bogdahn, U.; et al. Radiotherapy plus Concomitant and Adjuvant Temozolomide for Glioblastoma. N. Engl. J. Med. 2005, 352 (10), 987-996.
(2) Wong, E. T.; Hess, K. R.; Gleason, M. J.; Jaeckle, K. A.; Kyritsis, A. P.; Prados, M. D.; Levin, V. A.; Yung, W. K. A. Outcomes and Prognostic Factors in Recurrent Glioma Patients Enrolled Onto Phase II Clinical Trials. J. Clin. Oncol. 1999, 17 (8), 2572-2572.
(3) Sarin, H.; Kanevsky, A. S.; Wu, H.; Brimacombe, K. R.; Fung, S. H.; Sousa, A. A.; Auh, S.; Wilson, C. M.; Sharma, K.; Aronova, M. A.; et al. Effective Transvascular Delivery of Nanoparticles across the Blood-Brain Tumor Barrier into Malignant Glioma Cells. J. Transl. Med. 2008, 6, 80.
(4) van Tellingen, O.; Yetkin-Arik, B.; de Gooijer, M. C.; Wesseling, P.; Wurdinger, T.; de Vries, H. E. Overcoming the Blood-brain Tumor Barrier for Effective Glioblastoma Treatment. Drug Resist. Updat. 2015, 19, 1-12.
(5) Neuwelt, E.; Abbott, N. J.; Abrey, L.; Banks, W. A.; Blakley, B.; Davis, T.; Engelhardt, B.; Grammas, P.; Nedergaard, M.; Nutt, J.; et al. Strategies to Advance Translational Research into Brain Barriers. Lancet Neurol. 2008, 7 (1), 84-96.
(6) Wang, D.; Lippard, S. J. Cellular Processing of Platinum Anticancer Drugs. Nat. Rev. Drug Discov. 2005, 4 (4), 307-320.
(7) Blommaert, F. A.; van Dijk-Knijnenburg, H. C. M.; Dijt, F. J.; den Engelse, L.; Baan, R. A.; Berends, F.; Fichtinger-Schepman, A. M. J. Formation of DNA Adducts by the Anticancer Drug Carboplatin: Different Nucleotide Sequence Preferences in Vitro and in Cells. Biochemistry (Mosc.) 1995, 34 (26), 8474-8480.
(8) Miller, R. P.; Tadagavadi, R. K.; Ramesh, G.; Reeves, W. B. Mechanisms of Cisplatin Nephrotoxicity. Toxins 2010, 2 (11), 2490-2518.
(9) Zheng, Y.-R.; Suntharalingam, K.; Johnstone, T. C.; Yoo, H.; Lin, W.; Brooks, J. G.; Lippard, S. J. Pt(IV) Prodrugs Designed to Bind Non-Covalently to Human Serum Albumin for Drug Delivery. J. Am. Chem. Soc. 2014,136 (24), 8790-8798.
(10) Dhar, S.; Gu, F. X.; Langer, R.; Farokhzad, O. C.; Lippard, S. J. Targeted Delivery of Cisplatin to Prostate Cancer Cells by Aptamer Functionalized Pt(IV) Prodrug-PLGA-PEG Nanoparticles. Proc. Natl. Acad. Sci. 2008,105 (45), 17356-17361.
(11) Abramkin, S.; M. Valiandi, S.; A. Jakupec, M.; Galanski, M.; Metzler-Nolte, N.; K. Keppler, B. Solid-Phase Synthesis of Oxaliplatin—TAT Peptide Bioconjugates. Dalton Trans. 2012, 41 (10), 3001-3005.
(12) Mukhopadhyay, S.; Barnés, C. M.; Haskel, A.; Short, S. M.; Barnes, K. R.; Lippard, S. J. Conjugated Platinum(IV)-Peptide Complexes for Targeting Angiogenic Tumor Vasculature. Bioconjug. Chem. 2008, 19 (1), 39-49.
(13) Wong, D. Y. Q.; Yeo, C. H. F.; Ang, W. H. Immuno-Chemotherapeutic Platinum(IV) Prodrugs of Cisplatin as Multimodal Anticancer Agents. Angew. Chem. Int. Ed Engl. 2014, 53 (26), 6752-6756.
(14) Gaviglio, L.; Gross, A.; Metzler-Nolte, N.; Ravera, M. Synthesis and in Vitro Cytotoxicity of Cis, Cis, Trans-Diamminedichloridodisuccinatoplatinum(Iv)-Peptide Bioconjugates. Metallomics 2012, 4 (3), 260-266.
(15) Yung, W. K.; Mechtler, L.; Gleason, M. J. Intravenous Carboplatin for Recurrent Malignant Glioma: A Phase II Study. J. Clin. Oncol. 1991, 9 (5), 860-864.
(16) Reardon, D. A.; Desjardins, A.; Peters, K. B.; Gururangan, S.; Sampson, J. H.; McLendon, R. E.; Herndon, J. E.; Bulusu, A.; Threatt, S.; Friedman, A. H.; et al. Phase II Study of Carboplatin, Irinotecan, and Bevacizumab for Bevacizumab Naïve, Recurrent Glioblastoma. J. Neurooncol. 2012,107 (1), 155-164.
(17) Francesconi, A. B.; Dupre, S.; Matos, M.; Martin, D.; Hughes, B. G.; Wyld, D. K.; Lickliter, J. D. Carboplatin and Etoposide Combined with Bevacizumab for the Treatment of Recurrent Glioblastoma Multiforme. J. Clin. Neurosci. 2010, 17 (8), 970-974.
(18) Fadzen, C. M.; Wolfe, J. M.; Cho, C.-F.; Chiocca, E. A.; Lawler, S. E.; Pentelute, B. L. Perfluoroarene-Based Peptide Macrocycles to Enhance Penetration Across the Blood-Brain Barrier. J. Am. Chem. Soc. 2017, 139 (44), 15628-15631.
(19) Mijalis, A. J.; Thomas Iii, D. A.; Simon, M. D.; Adamo, A.; Beaumont, R.; Jensen, K. F.; Pentelute, B. L. A Fully Automated Flow-Based Approach for Accelerated Peptide Synthesis. Nat. Chem. Biol. 2017, 13 (5), 464-466.
(20) Berger, G.; Grauwet, K.; Zhang, H.; Hussey, A. M.; Nowicki, M. O.; Wang, D. I.; Chiocca, E. A.; Lawler, S. E.; Lippard, S. J. Anticancer Activity of Osmium(VI) Nitrido Complexes in Patient-Derived Glioblastoma Initiating Cells and in Vivo Mouse Models. Cancer Lett. 2018, 416, 138-148.
(21) Williams, S. P.; Nowicki, M. O.; Liu, F.; Press, R.; Godlewski, J.; Abdel-Rasoul, M.; Kaur, B.; Fernandez, S. A.; Chiocca, E. A.; Lawler, S. E. Indirubins Decrease Glioma Invasion by Blocking Migratory Phenotypes in Both the Tumor and Stromal Endothelial Cell Compartments. Cancer Res. 2011, 71 (16), 5374-538

1. A conjugate comprising
C2-L2-Pt-L1-C1
wherein Pt is a platinum(IV) prodrug; L1, L2 are absent or a linker; C₁ is a first perfluoroaryl peptide; and C₂ is absent or a second perfluoroaryl peptide, wherein the first perfluroaryl peptide may be identical to or different from the second perfluoroaryl peptide.

2. The conjugate of claim 1, wherein the peptide comprises two cysteines.

3. The conjugate of claim 1, wherein the platinum(IV) prodrug is cisplatin with the addition of two axial ligands.

4. The conjugate of claim 1, wherein the perfluoroaryl peptide is represented by the following formula 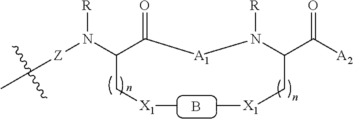
wherein
A₁ comprises 1-20 natural or unnatural amino acids;
A₂ is OH, NH₂, a carboxylate protecting group, a natural or unnatural amino acid, a peptide, an oligopeptide, a polypeptide, or a protein;
R is H or alkyl;
X₁ is S or NH;
Z is a natural or unnatural amino acid, a plurality of natural amino acids or unnatural amino acids, a peptide, an oligopeptide, a polypeptide;
n is 0, 1, 2, 3, 4, 5, or 6; and 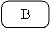
is a perfluoroaryl para-substituted diradical.

5. The conjugate of claim 4, wherein 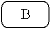
is 2,3,5,6-tetrafluorophenylene or 2,2′,3,3′,5,5′,6,6′-octafluoro-1,1′-biphenyl-4,4′-ene.

6. The conjugate of claim 1, wherein the conjugate is represented by the formula (SEQ ID NO: 7): 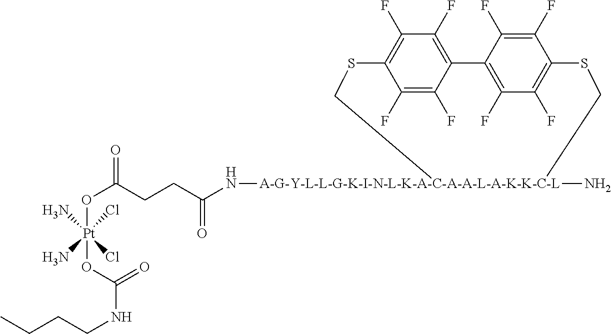
and pharmaceutically acceptable salts thereof.

7. The conjugate of claim 1, wherein the platinum is connected to the perfluoroaryl peptide at one axial position and has a —OC(═O)R₂ or —OC(═O)NHR₂ ligand at the other axial position, wherein R₂ is C₁-C₁₀ alkyl.

8. The conjugate of claim 1, wherein C2 is a second perfluoroaryl peptide.

9. The conjugate of claim 8, the conjugate is represented by the formula as follows, wherein each of the two amino acid sequences bound to the cisplatin group are represented by (SEQ ID NO: 8):

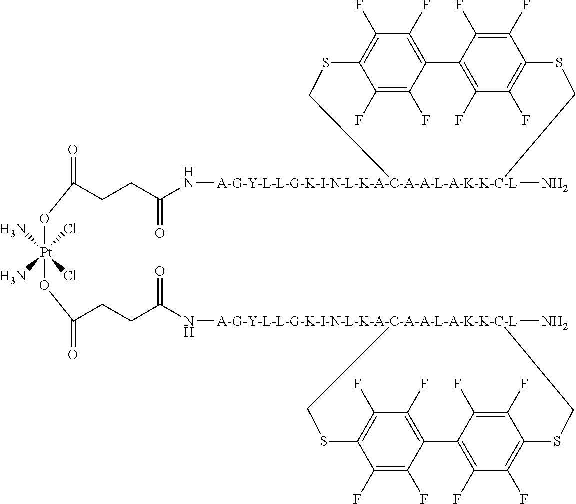

10. A pharmaceutical composition comprising the conjugate of any one of claims 1-9 and a pharmaceutical carrier.

11. A method of inhibiting growth of a tumor comprising contacting the tumor with an effective amount of the conjugate of any one of claims 1-9.

12. The method of claim 11, wherein the tumor is glioblastoma.

13. A method of treating cancer comprising administering a therapeutically effective amount of the conjugate of any one of claims 1-9.

14. The method of claim 13, wherein the cancer is glioblastoma.

15. A method of killing a cancer cell comprising contact the cancer cell with a therapeutically effective amount of the conjugate of any one of claims 1-9.

16. The method of claim 15, wherein the cancer cell is glioblastoma cell.

17. A method of making a platinum (IV) prodrug perfluoroaryl peptide conjugate comprising:
according to the following scheme, 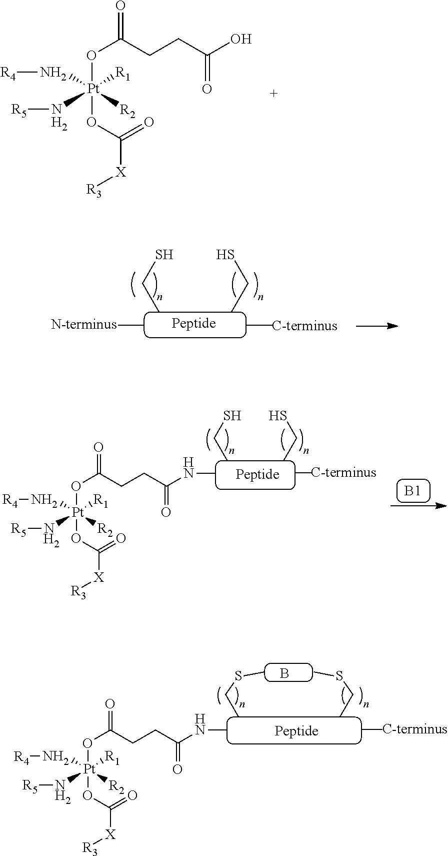
wherein
R₁ and R₂ each is C₁, or R₁ and R₂ are joined to form an oxalate or 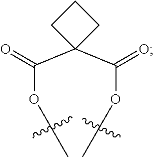 [SMILES: *OC(=O)C1(C(=O)O*)CCC1]
R₃ is selected from the group consisting of alkyl, cycloalkyl, heterocyclyl, aryl, and heteroaryl, wherein each of the alkyl, cycloalkyl, heterocyclyl, aryl, and heteroaryl groups optionally is substituted with one or more groups;
R₄ and R₅ are each H or together constitute a cyclohexyl ring;
n is 0, 1, 2, 3, 4, 5, or 6;
X is absent or NH; 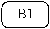
is a perfluoroaryl compound; and 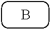
is a perfluoroaryl para-substituted diradical.

18. The method of claim 17, wherein 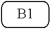
is hexafluorophenyl or decafluorobiphenyl.

19. The method of claim 17, wherein 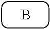
is 2,3,5,6-tetrafluorophenylene or 2,2′,3,3′,5,5′,6,6′-octafluoro-1,1′-biphenyl-4,4′-ene.
